(19) United States
(12) Patent Application Publication
(10) Pub. No.: US 2026/0131171 A1
(43) Pub. Date: May 14, 2026

(54) FOCUSED ULTRASOUND SURGICAL INSTRUMENT WHICH IS CAPABLE OF IMPLEMENTING SURFACE IRRADIATION OF SPIRAL PATH AND SURGICAL METHOD USING THE SAME

(71) Applicant: Shenb Co., Ltd., Seoul (KR)

(72) Inventor: Sunyoung KANG, Seoul (KR)

(21) Appl. No.: 18/960,536

(22) Filed: Nov. 26, 2024

(30) Foreign Application Priority Data
Nov. 12, 2024 (KR) 10-2024-0159990

(51) Int. Cl. A61N 7/00

(52) U.S. Cl. CPC A61N 7/00; A61N 2007/0034; A61N 2007/0086

(57) ABSTRACT

A surgical instrument for irradiating a focused ultrasound includes a main body, a driving unit which is provided in the main body to generate arbitrary rotational force, a support plate which is fixed into the main body by forming a spiral guide groove on any one surface and allows one side of the driving unit to penetrate to be supported, a traction unit which is coupled to one penetrating side of the driving unit to rotate in response to the rotational force generated in the driving unit and is movably coupled to the guide groove of the support plate to be pulled to draw a spiral path along the guide groove during the rotation, and a following unit which is coupled to one side of the traction unit to output a focused ultrasound while following the traction motion of the traction unit.

### CROSS-REFERENCE TO RELATED APPLICATIONS

This application claims the priority of Korean Patent Application No. 10-2024-0159990 filed on Nov. 12, 2024, in the Korean Intellectual Property Office, the disclosure of which is incorporated herein by reference.

### BACKGROUND

### Field

The present disclosure relates to a focused ultrasound surgical instrument which is capable of implementing surface irradiation of a spiral path which allows a transfer structure which includes solely a driving unit for generating power and moves an ultrasound output unit along a spiral path by a physical action to change a focus of a ultrasound to implement surface irradiation and simplify the structure so that more efficient production processes and a relatively reasonable unit prices may be expected, a failure rate may be significantly reduced, and a size of the device may be reduced to expand a surgical target area more widely and a surgical method using the same.

### Description of the Related Art

In the related art, a technology related to ultrasound generators has been known for using focused ultrasound generated by focusing ultrasound generated by an ultrasound generator to achieve skin beauty and treatment for therapeutic purposes.

Generally, the ultrasound generator is designed to be divided into a handpiece and a cartridge. The handpiece is a body of the ultrasound generator designed to be comfortable for a user to hold to use the ultrasound generator and the cartridge is a part which includes an ultrasound transducer which generates ultrasound and components required to drive the ultrasound transducer and is coupled to the handpiece.

As studies and developments are being conducted to irradiate a focused ultrasound on a skin of a subject in a wide area, the ultrasound generator configured by the handpiece and the cartridge is designed to have various modified structures and a surgical method corresponding thereto is also rapidly spreading in the medical beauty related industries.

That is, various techniques which implement surface irradiation of the ultrasound by means of a plurality of linear transfer means, rather than simply a linear transfer means with a single structure in the transducer of the related art are disclosed.

However, according to the technology of the related art, two or more driving units, such as a motor are required, which causes the complex structure and a cost burden, and an increased size of the device.

Specifically, due to the increased size of the device, there are limitations in that the treatment is applied only to a large area of the body, such as the abdomen, in terms of a skin area which is a subject to the treatment and it is difficult to apply a relatively narrow area, such as the face, so that the usage is limited.

Accordingly, there are increased needs for a focused ultrasound based skin beauty and treatment devices to which a technique which implements surface irradiation even with a relatively simple structure so that efficiency production processes and reasonable production unit prices may be expected and wide expansion of the surgical target area may be achieved with a reduced size of the device by simplifying the structure is applied.

### RELATED ART DOCUMENT

### Patent Document

(Patent Document 1) Korean Registered Patent Publication No. 10-1712024 (Title of Invention: Medical apparatus using high intensity focused ultrasound)

### SUMMARY

Accordingly, the present disclosure is contrived to solve the above-described problems and an object is to provide a focused ultrasound surgical instrument which is capable of implementing surface irradiation of a spiral path which allows a transfer structure which includes solely a driving unit for generating power and moves an ultrasound output unit along a spiral path by a physical action to change a focus of a ultrasound to implement surface irradiation and simplify the structure so that more efficient production processes and a relatively reasonable unit prices may be expected, a failure rate may be significantly reduced, and a size of the device may be reduced to expand a surgical target area more widely and a surgical method using the same.

However, technical objects to be achieved in the present invention are not limited to the aforementioned technical objects, and another not-mentioned technical object will be clearly understood by those skilled in the art from the description below.

In order to achieve the above-described objects, as a technical means, according to an aspect of the present disclosure, a focused ultrasound surgical instrument which is capable of implementing surface irradiation of a spiral path is a surgical instrument for irradiating a focused ultrasound including a main body, a driving unit which is provided in the main body to generate arbitrary rotational force, a support plate which is fixed into the main body by forming a spiral guide groove on any one surface and allows one side of the driving unit to penetrate to be supported, a traction unit which is coupled to one penetrating side of the driving unit to rotate in response to the rotational force generated in the driving unit and is movably coupled to the guide groove of the support plate to be pulled to draw a spiral path along the guide groove during the rotation, and a following unit which is coupled to one side of the traction unit to output a focused ultrasound while following the traction motion of the traction unit.

Further, the main body may be configured by a first housing; and a second housing which is detachably coupled to the first housing.

Further, the first housing may be a handpiece housing and the second housing may be a cartridge housing which is replaceable with the handpiece housing, the driving unit, the support plate, and the traction unit are disposed in the first housing and the following unit may be disposed in the second housing.

Further, the driving unit may include a motor which is provided in the first housing solely; and a motor shaft which is supported by penetrating a center of the support plate and rotates by the driving of the motor.

Further, the traction unit may include a first frame which is coupled to an end of the penetrated motor shaft to extend around a periphery of the support plate and rotate around the motor shaft in accordance with the rotation of the motor shaft; a second frame which is slidably coupled to the first frame perpendicularly, through a guide hole which penetrates in the extending direction of the first frame; a guider which protrudes from one end of the second frame in a direction perpendicular to the extending direction of the first frame so as to correspond to a guide groove of the support plate to be movably inserted into the guide groove; and a traction shaft which is provided at the other end of the second frame to form an axis line parallel to the motor shaft to be in contact with a part of the following unit when the first and second housings are coupled.

Further, when the first frame rotates, the guider generates stress between a surface of the first frame and a surface of the guide hole of the second frame while rotating in the spiral path along the guide groove to pull the second frame to slide along the surface of the first frame and when the second frame is pulled, the traction shaft is pulled by the guider integrally with the second frame and ultimately, pulls a part of the contacted following unit along the same spiral path as the guider.

Further, the following unit may include a transducer assembly which is configured by a bracket which is pulled while being in contact with the traction shaft of the traction unit to follow the traction motion of the traction shaft and an ultrasound transducer which is coupled to the bracket to be disposed to face the end portion of the second housing which is in contact with a skin of a subject to output a focused ultrasound in a predetermined frequency; a sealing member which is manufactured to have elasticity to movably support the transducer assembly to be spaced apart from the inner surface of the second housing and seals to form a sealed space in which the ultrasound transducer of the transducer assembly is accommodated in the second housing; an acoustic liquid which is filled in the sealed space to transmit the ultrasound output from the ultrasound transducer to the end portion of the second housing and reduce heat generated according to the ultrasound output of the ultrasound transducer; and a transmissive film which is provided in the end portion of the second housing to transmit the ultrasound transmitted by the acoustic liquid to the outside.

Further, the center of the transmissive film may form a straight line with the motor shaft located at the center of the support plate and the second frame may be bent toward the axial line of the motor shaft to align the axial line of the traction shaft to be close to the center of the support plate.

Further, the traction shaft may have a protrusion which protrudes to be narrower as it approaches the bracket and has a built-in magnetic body and the bracket may have an accommodation groove which is correspondingly formed to accommodate the protrusion of the traction shaft and interacts with the magnetic body of the protrusion when the first and second housings are decoupled or coupled, to be detachable by the magnetism with the traction shaft and a surface of the accommodation groove is formed of a magnetic material.

In the meantime, in order to achieve the above-described objects, as a technical means, according to another aspect of the present disclosure, a surgical method using a focused ultrasound surgical instrument which is capable of implementing surface irradiation of a spiral path is a surgical method using a surgical instrument for irradiating a focused ultrasound including a) a step of generating an arbitrary rotational force by a driving unit; b) a step of rotating a traction unit in response to the rotational force generated in the driving unit in the step a) and pulling the traction unit to draw a spiral path along a spiral guide groove formed on a support plate; and c) a step of outputting a focused ultrasound while a following unit follows the traction motion of the traction unit by the step b).

Further, the surgical method may further include: prior to the step a), a′) a step of coupling a first housing and a second housing in which the driving unit, the support plate, the traction unit, and the following unit are selectively disposed to form a main body.

Further, in the step a′), the driving unit, the support plate, and the traction unit are located in the first housing and the following unit is located in the second housing and in the step a′), the first housing and the second housing are detachably coupled.

Further, the first housing in the step a′) may be a handpiece housing and the second housing in the step a′) may be a cartridge housing which is replaceable with the handpiece housing.

Further, the step a) may include, a-1) a step of driving a motor of the driving unit; and a-2) a step of generating an arbitrary rotational force while a motor shaft of the driving unit rotates by the driving of the motor, and the motor in the step a-1) may be provided in the first housing solely and the motor shaft in the step a-2) may be supported by penetrating a center of the support plate.

Further, the step b) may include b-1) a step of rotating a first frame of the traction unit in response to the rotational force generated in the step a-2); b-2) a step of pulling the second frame to slide along the surface of the first frame by generating stress between a surface of the first frame and a surface of the guide hole of the second frame of the traction unit while the guider of the traction unit rotates in the spiral path along the guide groove when the first frame rotates in the step b-1); and b-3) a step of pulling the traction shaft of the traction unit integrally with the second frame by the guider when the second frame is pulled in the step b-2) and ultimately, pulling a part of the following unit along the same spiral path as the guider.

Further, in the step a′), a transmissive film of the following unit and the support plate are located concentrically and when the traction shaft is pulled in the step b-3), an axial line of the traction shaft is aligned by the second frame which is bent toward an axial line of the motor shaft to be close to the center of the support plate.

Further, in the step a′), when the first housing and the second housing are coupled, the traction shaft of the traction unit and a bracket of the following unit are in contact with each other to be closely fixed and the traction shaft in the step a′) may have a protrusion which protrudes to be narrower as it approaches the bracket and includes a magnetic body and the bracket in the step a′) may have an accommodation groove which is correspondingly formed to accommodate the protrusion of the traction shaft and interacts with the magnetic body of the protrusion, to be detachable by the magnetism with the traction shaft and a surface of the accommodation groove is formed of a magnetic material.

The focused ultrasound surgical instrument which is capable of implementing surface irradiation of a spiral path and the surgical method using the same according to the present disclosure provide effects that a driving unit for generating power is solely provided and a transfer structure which moves the following unit for outputting the ultrasound in the spiral path by a physical reaction with the support plate and the traction unit may change a focal point of a ultrasound wave to implement surface irradiation and the structure is simplified so that more efficient production processes and a relatively reasonable unit prices may be expected, a failure rate may be significantly reduced, and a size of the device may be reduced to expand a surgical target area more widely.

Further, according to the present disclosure, the second frame which is located to be biased to the support plate due to structure is bent toward the axis line of the motor shaft located at the center of the support plate to align the traction shaft located in the bent end portion of the second frame to be close to the center of the support plate. Accordingly, the transducer assembly which follows the movement of the traction shaft is prevented from being deviated from a transmissible area of the transmissive film, which is concentrically formed with the support plate, thereby ensuring higher reliability in terms of the efficiency according to a degree of irradiating the ultrasound actually.

Further, according to the present disclosure, a configuration that the traction shaft of the traction unit and the bracket of the following unit disposed in the first housing and the second housing which form the main body 110, respectively, are detachable by the magnetism allows more solid and closer fixing when the first and the second housing are coupled, thereby ensuring a higher reliability in terms of an accuracy in the traction and following operation.

A technical object to be achieved in the present disclosure is not limited to the aforementioned effects, and another not-mentioned effects will be obviously understood by those skilled in the art from the description below.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a cross-sectional view schematically illustrating an overall configuration of a focused ultrasound surgical instrument which is capable of implementing surface irradiation of a spiral path according to an exemplary embodiment of the present disclosure;

FIG. 2 is a plan view individually enlarging a support plate of the focused ultrasound surgical instrument of FIG. 1;

FIG. 3 is a perspective view individually enlarging a traction unit of the focused ultrasound surgical instrument of FIG. 1;

FIGS. 4A to 4C are operation status views illustrating a traction motion of a traction unit acting on a support plate of the focused ultrasound surgical instrument of FIG. 1; and

FIG. 5 is a flowchart illustrating a surgical method using a focused ultrasound surgical instrument which is capable of implementing surface irradiation of a spiral path according to an exemplary embodiment of the present disclosure.

### DETAILED DESCRIPTION OF THE EMBODIMENT

Hereinafter, exemplary embodiments of the present invention will be described more fully with reference to the accompanying drawings for those skilled in the art to easily implement the present invention. Description of the present invention is just an embodiment for structural and functional description so that the scope of the present invention is not interpreted to be limited by the embodiment described in the specification. That is, the embodiment may be modified in various forms so that it is understood that the scope of the present invention has equivalents which are capable of implementing the technical spirit. Further, it does not mean that the specific embodiment includes the object or effect proposed in the present invention or includes only the effect so that it is not understood that the scope of the present invention is limited thereby.

In the meantime, meanings of terms described in the present invention may be understood as follows.

The terms “first” or “second” are used to distinguish one component from the other component so that the scope should not be limited by these terms. For example, a first component may be referred to as a second component, and similarly, a second component may be referred to as a first component. It should be understood that, when it is described that an element is “connected” to another element, the element may be directly connected to the other element or connected to the other element through a third element. In contrast, it should be understood that, when it is described that an element is directly connected to another element, no element is present between the element and the other element. Other expressions which describe the relationship between components, that is, “between” and “directly between”, or “adjacent to” and “directly adjacent to” need to be interpreted by the same manner.

Unless the context apparently indicates otherwise, singular expressions should be understood to include plural expressions, and it should be understood that terms “include” or “have” indicate that a feature, a number, a step, an operation, a component, a part or the combination thereof described in the specification is present, but do not exclude a possibility of presence or addition of one or more other features, numbers, steps, operations, components, parts or combinations thereof, in advance.

Unless they are contrarily defined, all terms used herein have the same meaning as those generally understood by a person with ordinary skill in the art. Terms which are defined in a generally used dictionary should be interpreted to have the same meaning as the meaning in the context of the related art but are not interpreted as an ideally or excessively formal meaning if it is not clearly defined in the present invention.

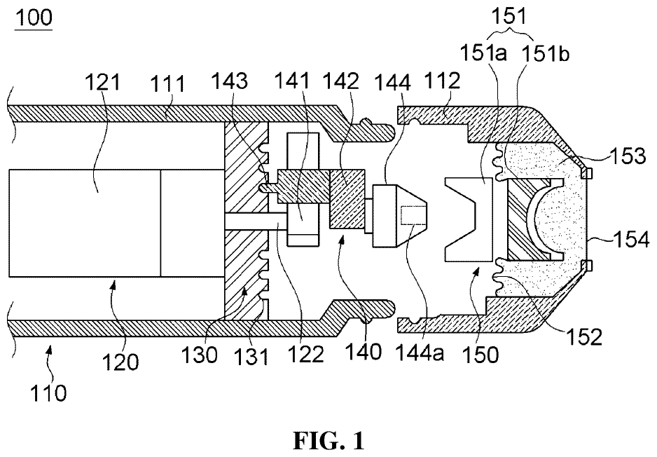

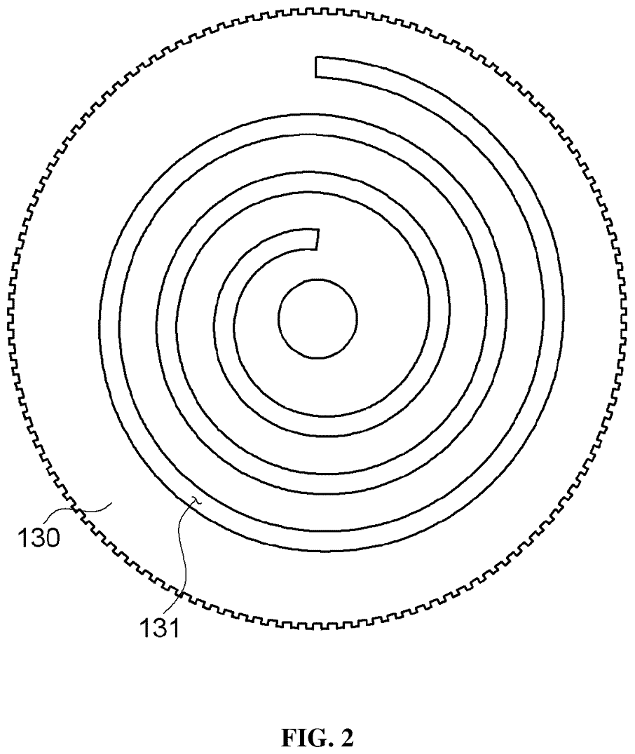

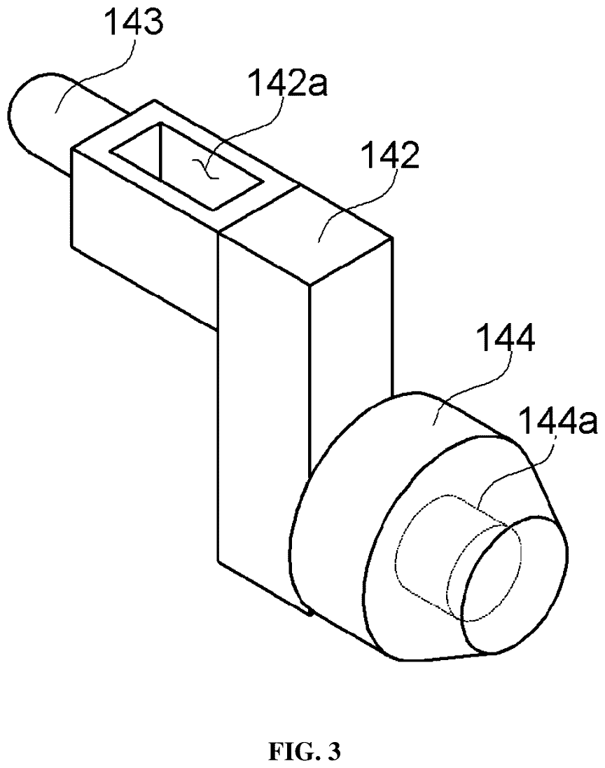

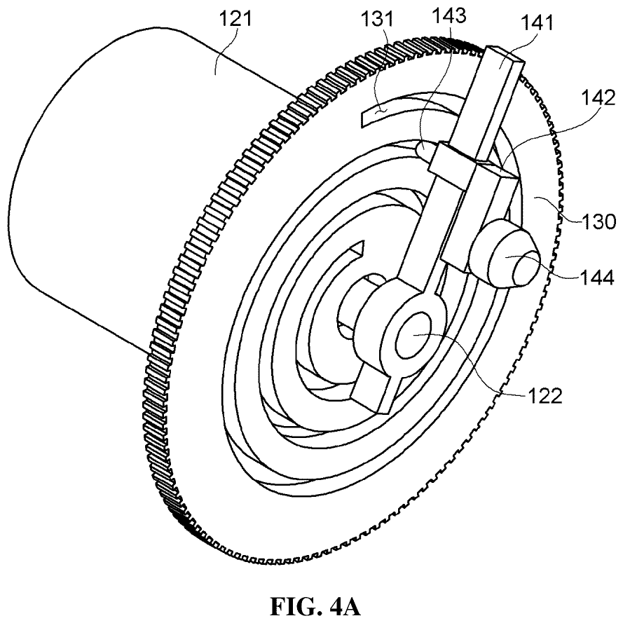

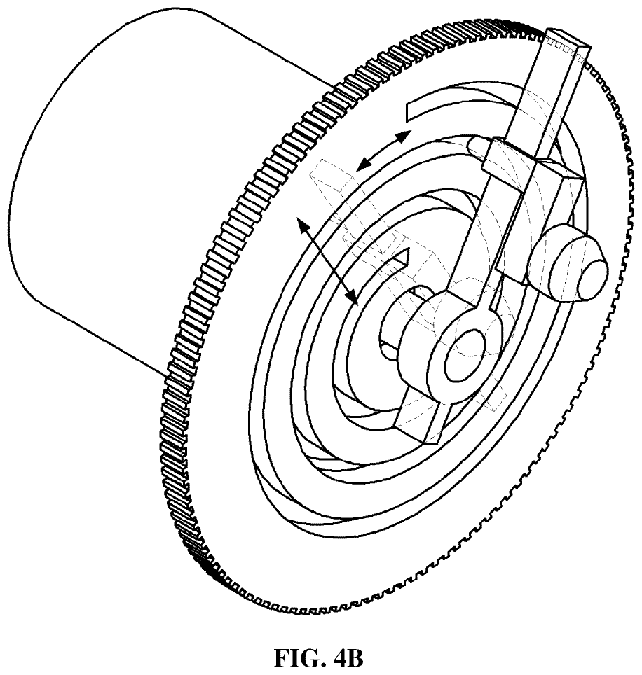

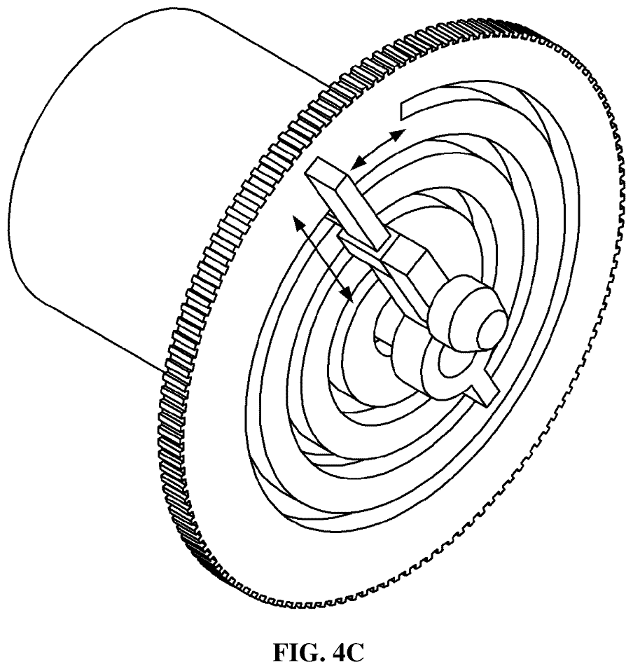

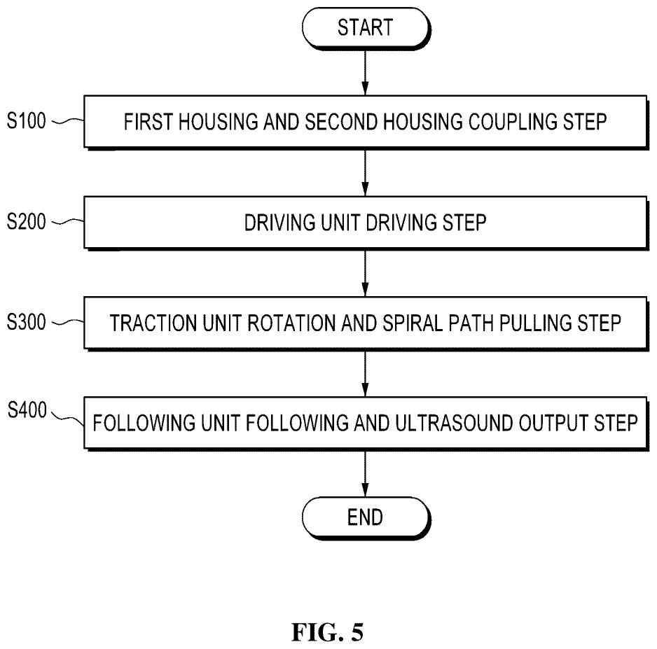

FIG. 1 is a cross-sectional view schematically illustrating an overall configuration of a focused ultrasound surgical instrument which is capable of implementing surface irradiation of a spiral path according to an exemplary embodiment of the present disclosure, FIG. 2 is a plan view individually enlarging a support plate of the focused ultrasound surgical instrument of FIG. 1, FIG. 3 is a perspective view individually enlarging a traction unit of the focused ultrasound surgical instrument of FIG. 1, FIGS. 4A to 4C are operation status views illustrating a traction motion of a traction unit acting on a support plate of the focused ultrasound surgical instrument of FIG. 1, and FIG. 5 is a flowchart illustrating a surgical method using a focused ultrasound surgical instrument which is capable of implementing surface irradiation of a spiral path according to an exemplary embodiment of the present disclosure.

As illustrated in FIGS. 1 to 3, a focused ultrasound surgical instrument 100 which is capable of implementing surface irradiation of a spiral path according to the present disclosure is a surgical instrument which irradiates a focused ultrasound and includes a main body 110, a driving unit 120, a support plate 130, a traction unit 140, a following unit 150.

The main body 110 covers each configuration of the present disclosure as a whole and is desirably formed with a cylindrical or polygonal cylindrical shape and further includes a grippable handle unit (not illustrated), but is not limited thereto.

According to the present disclosure, the main body 110 may be divided into a first housing 111 and a second housing 112 which is detachably coupled to the first housing 111.

The first housing 111 is desirably a handpiece housing and the second housing 112 is a cartridge housing which is replaceable with the above-described handpiece housing. The handpiece and the cartridge mentioned above employ a generally known technology so that a detailed description thereof will be omitted.

However, according to the exemplary embodiment of the present disclosure, in the first housing 111, the driving unit 120, the support plate 130, and the traction unit 140 are desirably disposed and the following unit 150 is desirably disposed in the second housing 112.

The driving unit 120 is a power source member which is provided in the main body 110 to generate an arbitrary rotational force and more desirably, is provided in the first housing 111 of the main body, and more specifically, may be configured to include a motor 121 and a motor shaft 122.

The motor 121 is desirably provided in the first housing 111 solely and various known driving means may be handled within the technical scope of the present disclosure which generates a rotational force while being driven by a power supply unit (not illustrated) which is detachably provided.

The above motor shaft 122 is an intuitive configuration which is obviously provided in the form of an axis on one side of the above-described motor 121 and may be rotated by the driving of the motor 121 while being supported by penetrating the center of the support plate 130.

The support plate 130 is a planar structure having a flat surface and according to the present disclosure, a spiral guide groove 131 is desirably formed on any one surface to be fixed in the first housing 111 of the main body 110 and allows one side of the driving unit 120 to penetrate to be supported.

At this time, the support plate 130 is disposed such that a direction in which a periphery (edge) expands is perpendicular to the length direction of the main body 110 and the guide groove 131 may be connected from the periphery (edge) of the support plate 130 to the center portion or from the center portion to the periphery (edge).

A size of the support plate 130 and a length and an interval of the guide grooves 131 may be designed in various forms by those skilled in the art within the technical scope of the present disclosure.

The traction unit 140 is coupled to one penetrating side of the above-described driving unit 120 to rotate in response to the rotational force generated in the driving unit 120 and is movably coupled to the guide groove 131 of the support plate 130 to be pulled to draw a spiral path along the guide groove 131 during the rotation.

The traction unit 140 may be configured to include a first frame 141, a second frame 142, a guider 143, and a traction shaft 144.

The first frame 141 is configured to be coupled to an end of the penetrated motor shaft 122 to rotate around the motor shaft 122 according to rotation of the motor shaft 112 while extending around the periphery of the support plate 130.

It is desirable that the second frame 142 is slidably coupled to the first frame 141 while being penetrated to the first frame 141 through a guide hole 142a which penetrates in the extending direction of the first frame 141.

The guider 143 protrudes from one end of the second frame 142 in a direction perpendicular to the extending direction of the first frame 141 so as to correspond to the guide groove 131 to be movably inserted into the guide groove 131.

The traction shaft 144 is provided at the other end of the second frame 142 to form an axial line parallel to the motor shaft 122 and is configured to come into contact with a part of the following unit 150 when the first and second housings 111 and 112 are coupled.

For example, referring to FIGS. 4A to 4C, according to the present disclosure, when the first frame 141 rotates, the guider 143 generates stress between a surface of the first frame 141 and a surface of the guide hole 142a of the second frame 142 while rotating in the spiral path along the guide groove 131 to pull the second frame 142 to slide along the surface of the first frame 141.

When the second frame 142 is pulled, the traction shaft 144 is pulled by the guider 143 integrally with the second frame 142 and ultimately, pulls a part of the contacted following unit 150 along the same spiral path as the guider 143.

The following unit 150 is a configuration which is coupled to one side of the above-described traction unit 140 to output a focused ultrasound while following the traction motion or the traction unit 140 and more particularly, is configured to include a transducer assembly 151, a sealing member 152, and an acoustic liquid 153, and a transmissive film 154.

The transducer assembly 151 is desirably configured by a bracket 151a which is pulled while being in contact with the traction shaft 144 of the traction unit 140 to follow the traction motion of the traction shaft 144 and an ultrasound transducer 151b which is coupled to the bracket 151a to be disposed to face the end portion of the second housing 112 which is in contact with the skin of the subject to output a focused ultrasound in a predetermined frequency.

The sealing member 152 is manufactured to have elasticity to movably support the transducer assembly 151 to be spaced apart from the inner surface of the second housing 112 and seals to form a sealed space in which the ultrasound transducer 151b of the transducer assembly 151 is accommodated in the second housing 112.

The sealing member 152 is desirably in the form of a corrugated tube which is manufactured to be elastic, but is not limited thereto.

The acoustic liquid 153 is an ultrasound transfer medium and is filled in the sealed space to serve to transmit the ultrasound output from the ultrasound transducer 151b to the end portion of the second housing 112 and reduce (cool) heat generated according to the ultrasound output of the ultrasound transducer 151b.

The transmissive film 154 is provided in the end portion of the second housing 112 to be configured to transmit the ultrasound transmitted by the acoustic liquid 153 to the outside and more desirably may be located in the center of the end portion of the second housing 112.

At this time, the “center” in the present disclosure refers to a center with respect to a thickness direction of the main body 110 and the center of the transmissive film 154 desirably forms a straight line with the motor shaft 122 located at the center of the support plate 130.

That is, the transmissive film 154 may be disposed concentrically with the support plate 130. Here, according to the exemplary embodiment of the present disclosure, the second frame 142 is bent toward the axial line of the motor shaft 122 to align the axial line of the traction shaft 144 to be close to the center of the support plate 130.

The bent shape of the second frame 142 may prevent the traction shaft 144 and the transducer assembly 151 pulled by the traction shaft from being deviated from the transmissible area of the transmissive film 154 in consideration of the ultrasound focus which is ultimately output.

In the meantime, the traction shaft 144 desirably has a protrusion (not illustrated) which protrudes to be narrower as it approaches the bracket 151a and has a built-in magnetic body 144a. The bracket 151a desirably has an accommodation groove (not denoted with a reference numeral) which is correspondingly formed to accommodate the protrusion of the traction shaft 144 and interacts with the magnetic body 144a of the protrusion when the first and second housings 111 and 112 are decoupled or coupled, to be detachable by the magnetism with the traction shaft 144. The surface of the accommodation groove is formed of a magnetic material.

Next, referring to FIG. 5, a surgical method using the focused ultrasound surgical instrument 100 which is capable of implementing surface irradiation of a spiral path configured as described above is a surgical method using a surgical instrument for irradiating focused ultrasound and includes a first housing and second housing coupling step S100, a driving unit driving step S200, a traction unit rotation and spiral path pulling step S300, and a following unit following and ultrasound output step S400.

In the first housing and second housing coupling step S100, as a step of preparing a treatment, the main body 110 is formed by coupling the first housing 111 and the second housing 112 in which the driving unit 120, the support plate 130, the traction unit 140, and the following unit 150 are selectively disposed.

The placement in the first housing and second housing coupling step S100 is performed such that the driving unit 120, the support plate 130, and the traction unit 140 are located in the first housing 111 and the following unit 150 is located in the second housing 112. More desirably, the transmissive film 154 of the following unit 150 and the support plate 130 are concentrically located.

Further, the coupling in the first housing and second housing coupling step S100 is performed such that the first housing 111 and the second housing 112 are separated from each other.

Further, in the first housing and second housing coupling step S100, the first housing 111 is desirably a handpiece housing and the second housing 112 is desirably a cartridge housing which is formed to be replaceable with the handpiece housing.

According to the present disclosure, in the first housing and second housing coupling step S100, when the first housing 111 and the second housing 112 are coupled, the traction shaft 144 of the traction unit 140 and the bracket 151a of the following unit 150 are in contact with each other to be closely fixed.

In the first housing and second housing coupling step S100, desirably, the traction shaft 144 has a protrusion (not denoted with a reference numeral) which protrudes to be narrower as it approaches the bracket 151a and has a built-in magnetic body 144a. The bracket 151a desirably has an accommodation groove (not denoted with a reference numeral) which is correspondingly formed to accommodate the protrusion of the traction shaft 144 and interacts with the magnetic body 144a of the protrusion to be detachable by the magnetism with the traction shaft 144. The surface of the accommodation groove is formed of a magnetic material.

The driving unit driving step S200 is a step in which the driving unit 120 generates an arbitrary rotational force and more specifically, includes a motor driving step (not denoted with a reference numeral) of driving the motor 121 of the driving unit 120 and a shaft rotation step (not dented with a reference numeral) of generating an arbitrary rotational force while the motor shaft 122 of the driving unit 120 rotates by driving the motor 121.

Here, the motor 121 of the motor driving step is provided in the first housing 111 solely and the motor shaft 122 of the shaft rotation step may be supported while penetrating the center of the support plate 130.

In the traction unit rotation and spiral path pulling step S300, the traction unit 140 rotates in response to the rotational force generated in the driving unit 120 through the driving unit driving step S200 and is pulled to draw a spiral path along the spiral guide groove 131 formed in the support plate 130.

To be more specific, the traction unit rotation and spiral path pulling step S300 may be configured to include a first frame rotation step (not denoted with a reference numeral), a guider pulling step (not denoted with a reference numeral)), and a following unit pulling step (not denoted with a reference numeral).

In the first frame rotation step, the first frame 141 of the traction unit 140 rotates in response to the rotational force generated in the shaft rotation step.

In the guider pulling step, the guider 143 of the traction unit 140 rotates in the spiral path along the guide groove 131 when the first frame 141 rotates in the first frame rotation step to generate a stress between a surface of the guide hole 142a formed in the second frame 142 of the traction unit 140 and a surface of the first frame 141 to pull the second frame 142 to slide along the surface of the first frame 141.

In the following unit pulling step, when the second frame 142 is pulled by the above-described guider pulling step, the traction shaft 144 of the traction unit 140 is pulled by the guider 143 integrally with the second frame 142 and ultimately, pulls a part of the following unit 150 along the same spiral path as the guider 143.

When the traction shaft 144 is pulled in the following unit pulling step, it is desirable that the traction shaft 144 is pulled by aligning the axial line of the traction shaft 144 by the second frame 142 bent toward the axial line of the motor shaft 122 to be close to the center of the support plate 130.

In the following unit following and ultrasound output step S400, the following unit 150 follows the traction motion of the traction unit 140 by the above-described traction unit rotation and spiral path pulling step S300 to output a focused ultrasound.

Accordingly, according to the focused ultrasound surgical instrument which is capable of implementing surface irradiation of a spiral path and the surgical method using the same according to the present disclosure, a transfer structure which includes a driving unit for generating power solely and moves the following unit 150 for outputting the ultrasound in the spiral path by a physical reaction with the support plate 130 and the traction unit 140 changes a focal point of a ultrasound wave to implement surface irradiation and the structure is simplified so that more efficient production processes and a relatively reasonable unit prices may be expected, a failure rate may be significantly reduced, and a size of the device may be reduced to expand a surgical target area more widely.

Further, according to the present disclosure, the second frame 142 which is located to be biased to the support plate 130 due to the structure is bent toward the axis line of the motor shaft 122 located at the center of the support plate 130 to align the traction shaft 144 located in the bent end portion of the second frame 142 to be close to the center of the support plate 130. Accordingly, the transducer assembly 151 which follows the movement of the traction shaft 144 is prevented from being deviated from a transmissible area of the transmissive film 154 which is concentrically formed with the support plate 130, thereby ensuring higher reliability in terms of the efficiency according to a degree of irradiating the ultrasound actually.

Furthermore, according to the present disclosure, the configuration that the traction shaft 144 of the traction unit 140 and the bracket 151a of the following unit 150 which are disposed in the first housing 111 and the second housing 112 which form the main body 110, respectively, are detachable by the magnetism allows more solid and closer fixing when the first and second housings 111 and 112 are coupled, to ensure higher reliability in terms of accuracy in the traction and following operation.

As described above, the detailed description of the exemplary embodiments of the disclosed present disclosure is provided such that those skilled in the art implement and carry out the present disclosure. While the disclosure has been described with reference to the preferred embodiments, it will be understood by those skilled in the art that various changes and modifications of the present disclosure may be made without departing from the spirit and scope of the disclosure. For example, those skilled in the art may use configurations disclosed in the above-described exemplary embodiments by combining them with each other. Therefore, the present invention is not intended to be limited to the above-described exemplary embodiments but to assign the widest scope consistent with disclosed principles and novel features.

The present disclosure may be implemented in another specific form within the scope without departing from the spirit and essential feature of the present disclosure. Therefore, the detailed description should not restrictively be analyzed in all aspects and should be exemplarily considered. The scope of the present disclosure should be determined by rational interpretation of the appended claims and all changes are included in the scope of the present disclosure within the equivalent scope of the present disclosure. The present disclosure is not intended to be limited to the above-described exemplary embodiments but to assign the widest scope consistent with disclosed principles and novel features. Further, claims having no clear quoting relation in the claims are combined to configure the embodiment or may be included as new claims by correction after application.

What is claimed is:

1. A focused ultrasound surgical instrument which is capable of implementing surface irradiation of a spiral path which is a surgical instrument for irradiating a focused ultrasound, comprising:
a main body;
a driving unit which is provided in the main body to generate arbitrary rotational force;
a support plate which is fixed into the main body by forming a spiral guide groove on any one surface and allows one side of the driving unit to penetrate to be supported;
a traction unit which is coupled to one penetrating side of the driving unit to rotate in response to the rotational force generated in the driving unit and is movably coupled to the guide groove of the support plate to be pulled to draw a spiral path along the guide groove during the rotation; and
a following unit which is coupled to one side of the traction unit to output a focused ultrasound while following the traction motion of the traction unit.

2. The focused ultrasound surgical instrument according to claim 1, wherein the main body includes:
a first housing; and
a second housing which is detachably coupled to the first housing.

3. The focused ultrasound surgical instrument according to claim 2, wherein the first housing is a handpiece housing, and the second housing is a cartridge housing which is replaceable with the handpiece housing, the driving unit, the support plate, and the traction unit are disposed in the first housing, and the following unit is disposed in the second housing.

4. The focused ultrasound surgical instrument according to claim 3, wherein the driving unit includes:
a motor which is provided in the first housing solely; and
a motor shaft which is supported by penetrating a center of the support plate and rotates by the driving of the motor.

5. The focused ultrasound surgical instrument according to claim 4, wherein the traction unit includes:
a first frame which is coupled to an end of the penetrated motor shaft to extend around a periphery of the support plate and rotate around the motor shaft in accordance with the rotation of the motor shaft;
a second frame which is slidably coupled to the first frame perpendicularly, through a guide hole which penetrates in the extending direction of the first frame;
a guider which protrudes from one end of the second frame in a direction perpendicular to the extending direction of the first frame so as to correspond to a guide groove of the support plate to be movably inserted into the guide groove; and
a traction shaft which is provided at the other end of the second frame to form an axis line parallel to the motor shaft to be in contact with a part of the following unit when the first and second housings are coupled.

6. The focused ultrasound surgical instrument according to claim 5, wherein when the first frame rotates, the guider generates stress between a surface of the first frame and a surface of the guide hole of the second frame while rotating in the spiral path along the guide groove to pull the second frame to slide along the surface of the first frame and when the second frame is pulled, the traction shaft is pulled by the guider integrally with the second frame and ultimately, pulls a part of the contacted following unit along the same spiral path as the guider.

7. The focused ultrasound surgical instrument according to claim 6, wherein the following unit includes:
a transducer assembly which is configured by a bracket which is pulled while being in contact with the traction shaft of the traction unit to follow the traction motion of the traction shaft and an ultrasound transducer which is coupled to the bracket to be disposed to face the end portion of the second housing which is in contact with a skin of a subject to output a focused ultrasound in a predetermined frequency;
a sealing member which is manufactured to have elasticity to movably support the transducer assembly to be spaced apart from the inner surface of the second housing and seals to form a sealed space in which the ultrasound transducer of the transducer assembly is accommodated in the second housing;
an acoustic liquid which is filled in the sealed space to transmit the ultrasound output from the ultrasound transducer to the end portion of the second housing and reduce heat generated according to the ultrasound output of the ultrasound transducer; and
a transmissive film which is provided in the end portion of the second housing to transmit the ultrasound transmitted by the acoustic liquid to the outside.

8. The focused ultrasound surgical instrument according to claim 7, wherein the center of the transmissive film forms a straight line with the motor shaft located at the center of the support plate and the second frame is bent toward the axial line of the motor shaft to align the axial line of the traction shaft to be close to the center of the support plate.

9. The focused ultrasound surgical instrument according to claim 7, wherein the traction shaft has a protrusion which protrudes to be narrower as it approaches the bracket and has a built-in magnetic body and the bracket has an accommodation groove which is correspondingly formed to accommodate the protrusion of the traction shaft and interacts with the magnetic body of the protrusion when the first and second housings are decoupled or coupled, to be detachable by the magnetism with the traction shaft and a surface of the accommodation groove is formed of a magnetic material.

10. A surgical method using a focused ultrasound surgical instrument which is capable of implementing surface irradiation of a spiral path, comprising:
a) a step of generating an arbitrary rotational force by a driving unit;
b) a step of rotating a traction unit in response to the rotational force generated in the driving unit in the step a) and pulling the traction unit to draw a spiral path along a spiral guide groove formed on a support plate; and
c) a step of outputting a focused ultrasound while a following unit follows the traction motion of the traction unit by the step b).

11. The surgical method using a focused ultrasound surgical instrument according to claim 10, further comprising:
prior to the step a), a′) a step of coupling a first housing and a second housing in which the driving unit, the support plate, the traction unit, and the following unit are selectively disposed to form a main body.

12. The surgical method using a focused ultrasound surgical instrument according to claim 11, wherein in the step a′), the driving unit, the support plate, and the traction unit are located in the first housing and the following unit is located in the second housing and
in the step a′), the first housing and the second housing are detachably coupled.

13. The surgical method using a focused ultrasound surgical instrument according to claim 12, wherein the first housing in the step a′) is a handpiece housing and the second housing in the step a′) is a cartridge housing which is replaceable with the handpiece housing.

14. The surgical method using a focused ultrasound surgical instrument according to claim 13, wherein the step a) includes:
a-1) a step of driving a motor of the driving unit; and
a-2) a step of generating an arbitrary rotational force while a motor shaft of the driving unit rotates by the driving of the motor, and
the motor in the step a-1) is provided in the first housing solely and the motor shaft in the step a-2) is supported by penetrating a center of the support plate.

15. The surgical method using a focused ultrasound surgical instrument according to claim 14, wherein the step b) includes:
b-1) a step of rotating a first frame of the traction unit in response to the rotational force generated in the step a-2);
b-2) a step of pulling the second frame to slide along the surface of the first frame by generating stress between a surface of the first frame and a surface of the guide hole of the second frame of the traction unit while the guider of the traction unit rotates in the spiral path along the guide groove when the first frame rotates in the step b-1); and
b-3) a step of pulling the traction shaft of the traction unit integrally with the second frame by the guider when the second frame is pulled in the step b-2) and ultimately, pulling a part of the following unit along the same spiral path as the guider.

16. The surgical method using a focused ultrasound surgical instrument according to claim 15, wherein in the step a′), a transmissive film of the following unit and the support plate are located concentrically and
when the traction shaft is pulled in the step b-3), an axial line of the traction shaft is aligned by the second frame which is bent toward an axial line of the motor shaft to be close to the center of the support plate.

17. The surgical method using a focused ultrasound surgical instrument according to claim 15, wherein in the step a′), when the first housing and the second housing are coupled, the traction shaft of the traction unit and a bracket of the following unit are in contact with each other to be closely fixed and
the traction shaft in the step a′) has a protrusion which protrudes to be narrower as it approaches the bracket and includes a magnetic body and the bracket in the step a′) has an accommodation groove which is correspondingly formed to accommodate the protrusion of the traction shaft and interacts with the magnetic body of the protrusion, to be detachable by the magnetism with the traction shaft and a surface of the accommodation groove is formed of a magnetic material.
